(19) United States
(12) Patent Application Publication
(10) Pub. No.: US 2022/0187291 A1
(43) Pub. Date: Jun. 16, 2022

(54) DEVICES AND METHODS FOR TEMPERATURE CORRECTION FOR LATERAL FLOW TESTING

(71) Applicant: Waters Technologies Corporation, Milford, MA (US)

(72) Inventors: Lingyun Chen, Westborough, MA (US); John Davenport, Milford, MA (US)

(73) Assignee: Waters Technologies Corporation, Milford, MA (US)

(21) Appl. No.: 17/544,235

(22) Filed: Dec. 7, 2021

Related U.S. Application Data
(60) Provisional application No. 63/123,618, filed on Dec. 10, 2020.

(51) Int. Cl. G01N 33/543

(52) U.S. Cl. CPC G01N 33/54388

(57) ABSTRACT

Temperature effects on results of a lateral flow assay can be compensated for by measuring an environmental temperature at a testing time and a testing location, performing a lateral flow assay at the testing time and testing location to obtain an original concentration value of a target substance, and generating a modified concentration value of the target substance. The modified concentration value is generated based on the original concentration value, by compensating for a difference between the environmental temperature and ambient temperature.

### CROSS-REFERENCE TO RELATED APPLICATION

This application claims priority and benefit to U.S. Provisional Patent Application No. 63/123,618, filed on Dec. 10, 2020, and entitled “Devices and Methods for Temperature Correction for Lateral Flow Testing”, the content of which is incorporated herein by reference in its entirety.

### TECHNICAL FIELD

The present disclosure relates generally to devices and methods for lateral flow testing. In particular, the present disclosure relates to devices and methods which can provide temperature correction for lateral flow tests.

### BACKGROUND

Lateral flow testing is used to assess concentrations of an analyte in solution. In the realm of food safety, lateral flow testing can be used to test for the presence of toxins, such as mycotoxins that can naturally occur on food products destined for animal or human consumption. The robustness of lateral flow testing devices allows for more active testing of products at the source, such as at farms or food-preparation facilities. According to traditional solutions, incubation is used to avoid temperature effects, which requires longer times and costlier equipment. In some cases, incubation can also require modified methods.

### SUMMARY

The present disclosure relates to devices and methods for compensating for temperature effects on results of a lateral flow assay.

In one aspect, the present disclosure relates to a method of compensating for temperature effects on results of a lateral flow assay. The method includes measuring an environmental temperature at a testing time and a testing location. The method also includes performing a lateral flow assay at the testing time and the testing location to obtain an original concentration value of a target substance. The method also includes generating a modified concentration value of the target substance, based on the original concentration value, by compensating for a difference between the environmental temperature and ambient temperature. In one embodiment, the modified concentration value is generated without incubation of a lateral flow test strip. In one embodiment, the testing location is a testing lab and the environmental temperature is a temperature of the testing lab at the testing time. In one embodiment, the environmental temperature is measured using a thermometer located at the testing time and at the testing location. In one embodiment, the thermometer is integral with a lateral flow reader. In one embodiment, the thermometer is a wireless thermometer in communication with a lateral flow reader.

According to another aspect, the present disclosure relates to a system for compensating for temperature effects on results of a lateral flow assay. The system includes a thermometer configured to measure an environmental temperature at a testing time and a testing location. The system also includes a lateral flow reader configured to receive a lateral flow test strip at the testing time and testing location to obtain an original concentration value of a target substance. The system also includes a computing device in communication with the thermometer and the lateral flow reader and configured to generate a modified concentration value of the target substance, based on the original concentration value, by compensating for a difference between the environmental temperature and ambient temperature. In one embodiment, the computing device generates the modified concentration value without incubation of the lateral flow test strip. In one embodiment, the testing location is a testing lab and the environmental temperature is a temperature of the testing lab at the testing time. In one embodiment, the computing device is integral with the lateral flow reader. In one embodiment, the thermometer is integral with a lateral flow reader. In one embodiment, the thermometer is a wireless thermometer in communication with the computing device.

According to another aspect, the present disclosure relates to a non-transitory machine-readable medium having instructions stored thereon, which when executed by a processor, cause the processor to perform a method of compensating for temperature effects on results of a lateral flow assay. The method includes measuring an environmental temperature at a testing time and a testing location. The method also includes performing a lateral flow assay at the testing time and the testing location to obtain an original concentration value of a target substance. The method also includes generating a modified concentration value of the target substance, based on the original concentration value, by compensating for a difference between the environmental temperature and ambient temperature. In one embodiment, the modified concentration value is generated without incubation of a lateral flow test strip. In one embodiment, the testing location is a testing lab and the environmental temperature is a temperature of the testing lab at the testing time. In one embodiment, the environmental temperature is measured using a thermometer located at the testing time and at the testing location. In one embodiment, the thermometer is integral with a lateral flow reader. In one embodiment, the thermometer is a wireless thermometer in communication with a lateral flow reader.

The devices and methods of the present disclosure provide several advantages over the prior art. For example, the devices and methods of the present disclosure can effectively adjust for temperature variations that can introduce error into a lateral flow test. This adjustment can be performed in real time, or alternatively performed after the fact based on the measured temperature data, the original concentration values generated by the lateral flow reader, and a compensation factor generated by a correlation between test strip results and different temperature changes. This adjustment can also be performed without incubation, which can simplify processes and avoid the need for expensive incubation equipment.

### BRIEF DESCRIPTION OF THE DRAWINGS

The invention will be more fully understood from the following detailed description taken in conjunction with the accompanying drawings, in which:

FIG. 1 shows a diagram of an example system for compensating for temperature effects on results of a lateral flow assay, according to an embodiment of the present disclosure.

FIG. 2A-2B illustrate flow charts of methods for compensating for temperature effects on results of a lateral flow assay, according to embodiments of the present disclosure.

FIG. 3 shows a diagram of an example network environment suitable for a distributed implementation of the processes described herein, according to an embodiment of the present disclosure.

FIG. 4 shows a block diagram of an example computing device that can be used to perform example processes and computations, according to principles of the present disclosure.

FIG. 5 shows a graph of uncorrected vs. corrected results of a lateral flow assay at various temperatures, according to an embodiment of the present disclosure.

The features and advantages of the present disclosure will become more apparent from the detailed description set forth below when taken in conjunction with the drawings.

### DETAILED DESCRIPTION

Devices and methods described herein provide devices and methods for compensating for temperature effects on results of a lateral flow assay. Lateral flow assay is widely used in food, environmental, and clinical diagnostics. As a rapid on-site screening tool, lateral flow assay differentiates from other sophisticated instrumental tests because it can be conducted in the field, instead of in a lab where the environment is well controlled. However, it has been observed that the results of a lateral flow assay are affected by the temperature of the environment where the assay is performed. The results of the assay are usually interpreted with the aid of a lateral flow test reader based on a calibration curve previously established. In some cases, when the assay is performed at a temperature different from the temperature the assay is calibrated, false results may occur.

According to embodiments of the present disclosure, the calibration curve can be modified mathematically in order to help mitigate the temperature effects of the results of the lateral flow assay.

The present disclosure introduces the concept of correcting the lateral flow assay results by compensating for the temperature effects mathematically. The actual temperature (T) of the environment where the assay is performed is measured by a thermometer. In some embodiments, the thermometer can be integrated to the lateral flow reader. In alternative embodiments, the thermometer can be external from the lateral flow reader, but in electronic communication with a computer processor that can perform the mathematically compensation. The temperature difference from the calibration temperature (T₀) is used to modify the final test result following a pre-defined algorithm.

Using a mycotoxin lateral flow test as an example, it was found that a plot of mycotoxin concentration against the temperature can be regarded as linear in the temperature range of interests (15 to 35 degree Celsius). The following formula can be used to compensate the temperature effects on the mycotoxin concentration results: where T is the lab temperature when the test is performed, T₀ is the calibration lab temperature, C is the modified result, C₀ is the original result without temperature compensation, and F is a constant factor pre-defined for the test. In various embodiments, the constant factor F can be different depending on the particular lateral flow test being performed.

C=C₀*[(T−T₀)*F+1] (1)

One skilled in the art will recognize that equation (1) is provided only as an example of how temperature variations can be compensated for. In different embodiments, a different algorithm can be used to compensate for temperature variations.

As used herein, the “environmental temperature” or the temperature of the “testing environment” is used to describe the temperature of the environment where a test is performed. This is distinct from the “ambient temperature,” which is a standard temperature (e.g. 25° C. or “room temperature” or “calibration temperature”) at which a calibration curve may be generated. In other words, the environmental temperature corresponds to the temperature that the test strip was exposed to during its development time (generally 2 to 10 minutes).

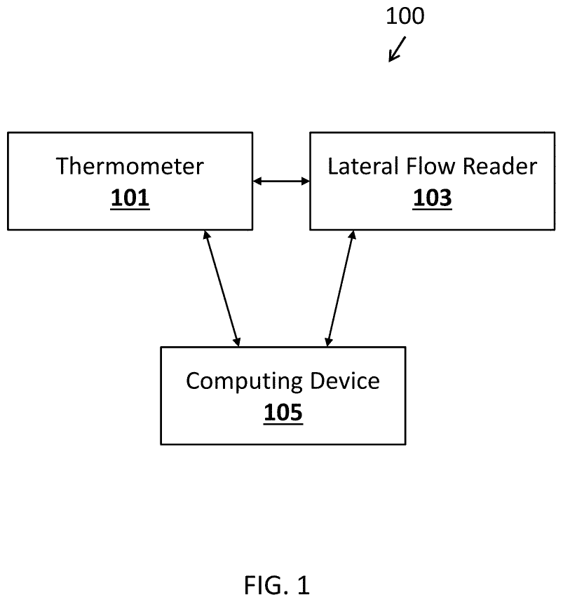

FIG. 1 shows a diagram of an example system for compensating for temperature effects on results of a lateral flow assay, according to an embodiment of the present disclosure. In this example embodiments, the system 100 includes a thermometer 101 configured to measure the temperature of a testing environment at a particular testing time. The system 100 also includes a lateral flow reader 103 that can be configured to receive a lateral flow test strip, and a computing device 105 that can be in communication with both the thermometer 101 and the lateral flow reader 103. As discussed above, the computing device can adjust for various temperature readings of the environment where the test is being performed. This corrects for potential errors that can be introduced due to temperature sensitivities of a lateral flow test. In general, lateral flow test strips used in connection with system 100 need not be thermally monitored and/or conditioned (i.e., incubated). That is, one of the advantages of the present technology is the ability to use lateral flow test strip at various environmental conditions and temperatures without the need to incubate or thermally regulate the test strip prior to and/or during a lateral flow assay.

The lateral flow reader 103 can be any device that accepts lateral flow strips and detects/indicates results from a lateral flow assay. A non-limiting example of such a lateral flow reader includes a mycotoxin testing reader, such as, Vertu lateral flow reader commercially available from Vicam, Milford, Mass. In some embodiments, thermometer 101 can be incorporated physically into the lateral flow reader 103. In other embodiments, thermometer 101 is a separate device from reader 103—however both thermometer 101 and reader 103 are in electronic communication, possibly through computing device 105 to share temperature information.

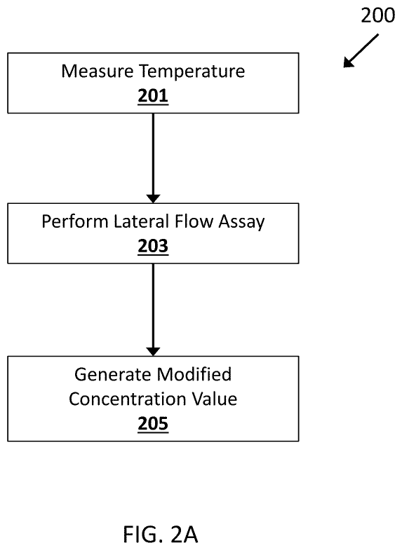

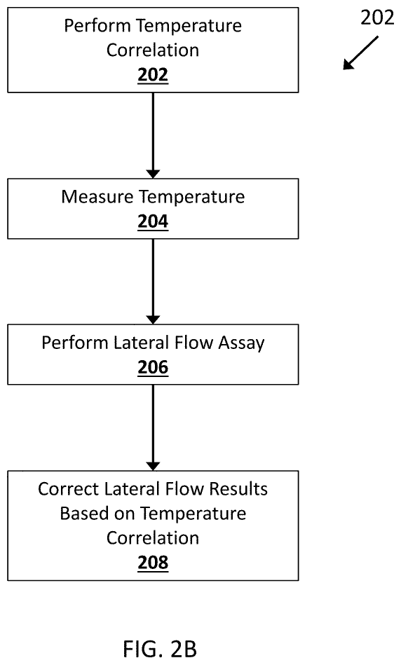

FIG. 2A-2B illustrate flow charts of methods for compensating for temperature effects on results of a lateral flow assay, according to embodiments of the present disclosure.

With reference to FIG. 2A, a method 200 can begin at operation 201 with measuring an environmental temperature at a testing time and a testing location. In some embodiments, the testing location is a testing lab and the environmental temperature is a temperature of the testing lab at the particular testing time. In alternative embodiments, the testing location can be outside of a laboratory. In some embodiments, the environmental temperature is measured using a thermometer located at the testing time and at the testing location. The thermometer may be integral with a lateral flow reader. However, in some embodiments it is important that the thermometer measure the environmental temperature of the space where the test is performed, and not the temperature of the electronic components of the lateral flow reader. In alternative embodiments, the thermometer can be a wireless thermometer in communication with a lateral flow reader, or a computing device.

At operation 203, the method continues with performing a lateral flow assay at the testing time and the testing location to obtain an original concentration value of a target substance. As discussed above, the concentration value measured can vary based on the environmental temperature where the test is performed. Thus, the original concentration value obtained at operation 203, which is based on a calibration curve generated at a potentially different temperature value (ambient temperature), may need to be adjusted or modified.

At operation 205, the modified concentration value is generated. In some embodiments, the modified concentration value is generated, based on the original concentration value, by compensating for a difference between the environmental temperature where the test is performed and ambient temperature. For example, the calibration curve used to generate concentration values from a lateral flow test is typically generated at ambient temperature (e.g. 25° C.). When a test is performed at a temperature other than this ambient temperature, the concentration value generated by the lateral flow reader can be off. Rather than solving this problem using incubation of the lateral flow test strip, the modified concentration value is generated mathematically.

With reference to FIG. 2B, method 202 begins at operation 202 with performing a temperature study that correlates test strip results with different temperatures. Once this correlation can be determined, it is possible that a mathematical equation could correct the outputted results by a particular factor (e.g., factor F, above). Once a factor can be determined, it is possible to correct for the difference in temperatures between when the calibration curve is generated and the test is performed. In contrast to incubation techniques, faster test results without the need for incubation equipment can be provided.

At operation 204, the method continues with measuring an environmental temperature at a testing time and a testing location. In some embodiments, the environmental temperature is measured using a thermometer located at the testing time and at the testing location. The thermometer may be integral with a lateral flow reader. However, in some embodiments it is important that the thermometer measure the environmental temperature of the space where the test is performed, and not the temperature of the electronic components of the lateral flow reader. In alternative embodiments, the thermometer can be a wireless thermometer in communication with a lateral flow reader, or a computing device.

At operation 206, the method continues with performing a lateral flow assay at the testing time and the testing location to obtain an original concentration value of a target substance. As discussed above, the concentration value measured can vary based on the environmental temperature where the test is performed. Thus, the original concentration value obtained at operation 206, which is based on a calibration curve generated at a potentially different temperature value (ambient temperature), may need to be adjusted or modified.

At operation 208, the modified concentration value is generated. In some embodiments, the modified concentration value is generated, based on the original concentration value, by compensating for a difference between the environmental temperature where the test is performed, and ambient temperature. For example, the calibration curve used to generate concentration values from a lateral flow test is typically generated at ambient temperature (e.g. 25° C.). When a test is performed at a temperature other than this ambient temperature, the concentration value generated by the lateral flow reader can be off. Rather than solving this problem using incubation of the lateral flow test strip, the modified concentration value is generated mathematically. The modified concentration value can be generated in real time, in some embodiments.

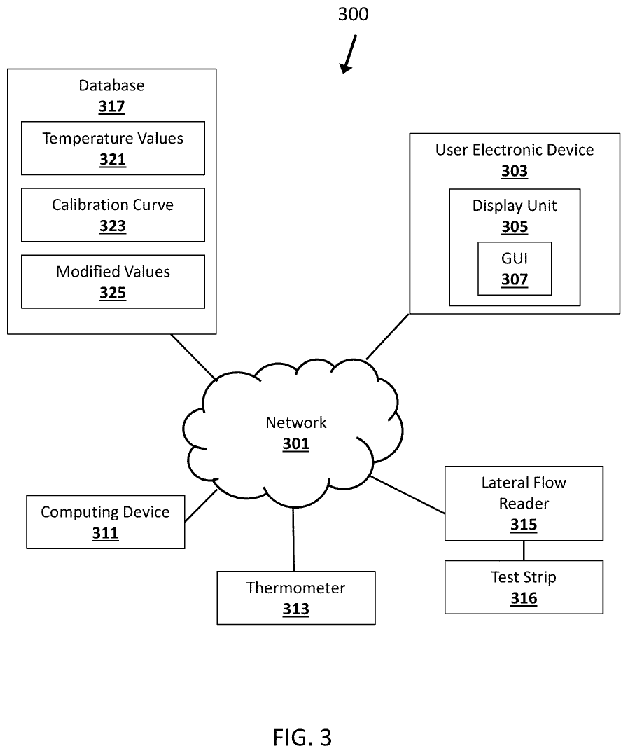

FIG. 3 illustrates a network diagram depicting a system 300 suitable for a distributed implementation of example systems described herein. The system 300 can include a network 301, a user electronic device 303, a computing device 311, thermometer 313, lateral flow reader 315, test strip 316, and database 317. As will be appreciated, the computing device 311 can be local or remote servers, and various distributed or centralized configurations may be implemented, and in some embodiments a single server can be used. In exemplary embodiments, the computing device 311 can include one or more modules, which can implement one or more of the processes described herein, or portions thereof, with reference to any one or more of FIGS. 2A-2B. In alternative embodiments, the computing device 311 can be integral with the lateral flow reader 315. The user electronic device 303 and computing device 311 can communicate with each other, and with the lateral flow reader 315, thermometer 313, and database 317 to transmit and receive data (such as, but not limited to, temperature values 321, calibration curve data 323, and the modified values 325 corresponding to the modified lateral flow test results), and implement the processes described above.

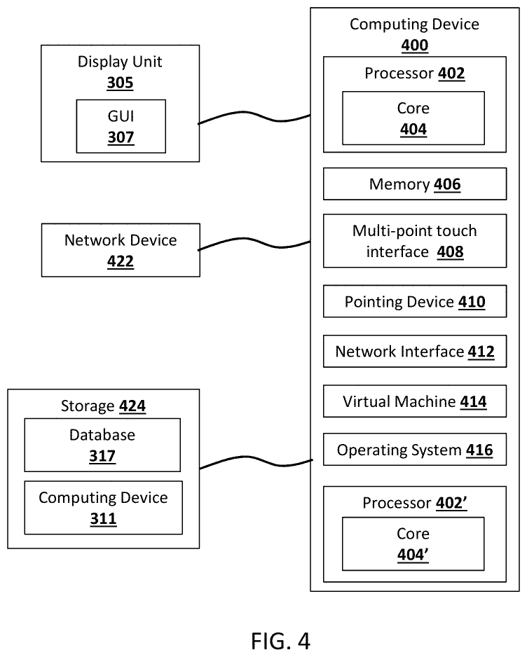

In exemplary embodiments, the user electronic device 303 may include a display unit 305, which can display a GUI 307 to a user of the device 303 such that the user can view a rendered graphic icon, visual display, or type of other signal used to indicate the lateral flow test results and/or the modified lateral flow test results. The user electronic device 303 may include, but is not limited to, smartphones, tablets, laptops, computers, general purpose computers, Internet appliances, hand-held devices, wireless devices, wearable computers, cellular or mobile phones, portable digital assistants (PDAs), desktops, and the like. The user electronic device 303 may include some or all components described in relation to computing device 400 shown in FIG. 4. The user electronic device 303 may connect to network 301 via a wired or wireless connection.

In exemplary embodiments, the user electronic device 303, computing device 311, thermometer 313, lateral flow reader 315, and database 317 may be in communication with each other via a communication network 301. The communication network 301 may include, but is not limited to, the Internet, an intranet, a LAN (Local Area Network), a WAN (Wide Area Network), a MAN (Metropolitan Area Network), a wireless network, an optical network, and the like.

FIG. 4 is a block diagram of an exemplary computing device 400 that can be used in the performance of any of the example methodologies according to the principles described herein (including example methodologies associated with any one or more of FIGS. 2A-2B). The computing device 400 includes one or more non-transitory computer-readable media for storing one or more computer-executable instructions (such as but not limited to software or firmware) for implementing any example method according to the principles described herein (including example methodologies associated with any one or more of FIGS. 2A-2B). The non-transitory computer-readable media can include, but are not limited to, one or more types of hardware memory, non-transitory tangible media (for example, one or more magnetic storage disks, one or more optical disks, one or more USB flash drives), and the like.

For example, memory 406 included in the computing device 400 can store computer-readable and computer-executable instructions or software for implementing exemplary embodiments and programmed to perform processes described above in reference to any one or more of FIGS. 2A-2B. The computing device 400 also includes processor 402 (and associated core 404), and optionally, one or more additional processor(s) 402′ and associated core(s) 404′ (for example, in the case of computer systems having multiple processors/cores), for executing computer-readable and computer-executable instructions or software stored in the memory 406 and other programs for controlling system hardware. Processor 402 and processor(s) 402′ can each be a single core processor or multiple core (404 and 404′) processor.

Virtualization can be employed in the computing device 400 so that infrastructure and resources in the computing device can be shared dynamically. A virtual machine 414 can be provided to handle a process running on multiple processors so that the process appears to be using only one computing resource rather than multiple computing resources. Multiple virtual machines can also be used with one processor.

Memory 406 can be non-transitory computer-readable media including a computer system memory or random access memory, such as DRAM, SRAM, EDO RAM, and the like. Memory 406 can include other types of memory as well, or combinations thereof.

A user can interact with the computing device 400 through a display unit 305, such as a touch screen display or computer monitor, which can display one or more user interfaces (GUI) 307 that can be provided in accordance with exemplary embodiments. The computing device 400 can also include other I/O devices for receiving input from a user, for example, a keyboard or any suitable multi-point touch interface 408, a pointing device 410 (e.g., a pen, stylus, mouse, or trackpad). The keyboard 408 and the pointing device 410 can be coupled to the display unit 305. The computing device 400 can include other suitable conventional I/O peripherals.

The computing device 400 can also include one or more storage devices 424, such as a hard-drive, CD-ROM, or other non-transitory computer readable media, for storing data and computer-readable instructions and/or software, such as a computing device 311 that can implement exemplary embodiments of the methodologies and systems as taught herein, or portions thereof. Exemplary storage device 424 can also store one or more databases 317 for storing any suitable information required to implement exemplary embodiments. The databases can be updated by a user or automatically at any suitable time to add, delete, or update one or more items in the databases.

The computing device 400 can include a network interface 412 configured to interface via one or more network devices 422 with one or more networks, for example, Local Area Network (LAN), Wide Area Network (WAN) or the Internet through a variety of connections including, but not limited to, standard telephone lines, LAN or WAN links (for example, 802.11, T1, T3, 56kb, X.25), broadband connections (for example, ISDN, Frame Relay, ATM), wireless connections, controller area network (CAN), or some combination of any or all of the above. The network interface 412 can include a built-in network adapter, network interface card, PCMCIA network card, card bus network adapter, wireless network adapter, USB network adapter, modem or any other device suitable for interfacing the computing device 400 to any type of network capable of communication and performing the operations described herein. Moreover, the computing device 400 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing device 400 can run any operating system 416, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 416 can be run in native mode or emulated mode. In an exemplary embodiment, the operating system 416 can be run on one or more cloud machine instances.

### EXAMPLES

In an example embodiment, a temperature correction can be calculated as outlined in the example below. Table 1 shows the original vs. corrected results for concentrations of deoxynivalenol (DON).

TABLE 1

| | | Temperature Degrees C. | | | | | |
|---|---|---|---|---|---|---|---|
| | | 14 | 18 | 22 | 26 | 30 | 34 |
| Original (Uncorrected) Results | | | | | | | |
| Expected | 0.5 ppm | 0.73 | 0.73 | 0.57 | 0.50 | 0.43 | 0.33 |
| Concentration of | 2.0 ppm | 2.73 | 2.53 | 2.27 | 1.83 | 1.70 | 1.20 |
| DON | | | | | | | |
| | Calibration | 22 | | | | | |
| | Temperature (C.) | | | | | | |
| | Factor | 0.042 | | | | | |
| Final (corrected) Results | | | | | | | |
| Expected | 0.5 ppm | 0.49 | 0.61 | 0.57 | 0.58 | 0.58 | 0.50 |
| Concentration of | 2.0 ppm | 1.81 | 2.11 | 2.27 | 2.14 | 2.27 | 1.80 |
| DON | | | | | | | |

The results in this example were corrected according to the following equation.

Original Result*[(ambient temperature (C)−calibration temperature (C))*Factor+1]=Corrected Result (2)

Taking the first result, which is 0.73, the formula would be:

0.73*[(14−22)*0.042+1]=0.49 (3)

This corrects the original result at 14° C. from an over estimated value of 0.73 ppm to a more accurate result of 0.49 ppm. The same can be shown across the range of temperatures and at different levels of toxins.

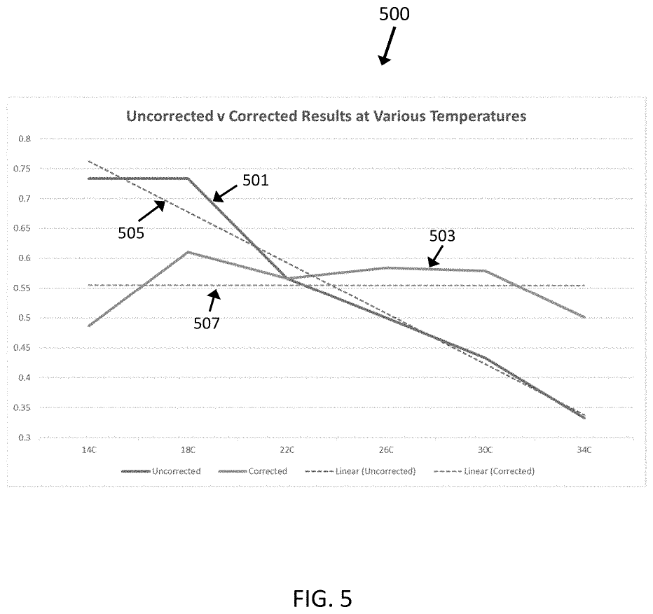

FIG. 5 shows a graph 500 of the uncorrected vs. corrected results at various temperatures. Specifically, the graph 500 shows a plot of the uncorrected results 501, as well as a linear representation 505 of the uncorrected results. The graph 500 also shows a plot of the corrected results 503, as well as a linear representation 507 of the corrected results.

In describing example embodiments, specific terminology is used for the sake of clarity. For purposes of description, each specific term is intended to at least include all technical and functional equivalents that operate in a similar manner to accomplish a similar purpose. Additionally, in some instances where a particular example embodiment includes a plurality of system elements, device components or method steps, those elements, components or steps can be replaced with a single element, component or step. Likewise, a single element, component or step can be replaced with a plurality of elements, components or steps that serve the same purpose. Moreover, while example embodiments have been shown and described with references to particular embodiments thereof, those of ordinary skill in the art will understand that various substitutions and alterations in form and detail can be made therein without departing from the scope of the disclosure. Further still, other aspects, functions and advantages are also within the scope of the disclosure.

Example flowcharts are provided herein for illustrative purposes and are non-limiting examples of methodologies. One of ordinary skill in the art will recognize that example methodologies can include more or fewer steps than those illustrated in the example flowcharts, and that the steps in the example flowcharts can be performed in a different order than the order shown in the illustrative flowcharts. Inventive embodiments of the present disclosure are directed to each individual feature, system, article, material, kit, and/or method described herein. In addition, any combination of two or more such features, systems, articles, materials, kits, and/or methodologies, if such features, systems, articles, materials, kits, and/or methodologies are not mutually inconsistent, is included within the inventive scope of the present disclosure.

What is claimed is:

1. A method of compensating for temperature effects on results of a lateral flow assay, comprising:
measuring an environmental temperature at a testing time and a testing location;
performing a lateral flow assay at the testing time and the testing location to obtain an original concentration value of a target substance; and
generating a modified concentration value of the target substance, based on the original concentration value, by compensating for a difference between the environmental temperature and ambient temperature.

2. The method of claim 1, wherein the modified concentration value is generated without incubation of a lateral flow test strip.

3. The method of claim 1, wherein the testing location is a testing lab and the environmental temperature is a temperature of the testing lab at the testing time.

4. The method of claim 1, wherein measuring the environmental temperature is performed using a thermometer located at the testing time and at the testing location.

5. The method of claim 4, wherein the thermometer is integral with a lateral flow reader.

6. The method of claim 4, wherein the thermometer is a wireless thermometer in communication with a lateral flow reader.

7. A system for compensating for temperature effects on results of a lateral flow assay, the system comprising:
a thermometer configured to measure an environmental temperature at a testing time and a testing location;
a lateral flow reader configured to receive a lateral flow test strip at the testing time and testing location to obtain an original concentration value of a target substance; and
a computing device in communication with the thermometer and the lateral flow reader and configured to generate a modified concentration value of the target substance, based on the original concentration value, by compensating for a difference between the environmental temperature and ambient temperature.

8. The system of claim 7, wherein the computing device generates the modified concentration value without incubation of the lateral flow test strip.

9. The system of claim 7, wherein the testing location is a testing lab and the environmental temperature is a temperature of the testing lab at the testing time.

10. The system of claim 7, wherein the computing device is integral with the lateral flow reader.

11. The system of claim 7, wherein the thermometer is integral with a lateral flow reader.

12. The system of claim 7, wherein the thermometer is a wireless thermometer in communication with the computing device.

13. A non-transitory machine-readable medium having instructions stored thereon, which when executed by a processor, cause the processor to perform a method of compensating for temperature effects on results of a lateral flow assay, the method comprising:
measuring an environmental temperature at a testing time and a testing location;
performing a lateral flow assay at the testing time and the testing location to obtain an original concentration value of a target substance; and
generating a modified concentration value of the target substance, based on the original concentration value, by compensating for a difference between the environmental temperature and ambient temperature.

14. The non-transitory machine-readable medium of claim 13, wherein the modified concentration value is generated without incubation of a lateral flow test strip.

15. The non-transitory machine-readable medium of claim 13, wherein the testing location is a testing lab and the environmental temperature is a temperature of the testing lab at the testing time.

16. The non-transitory machine-readable medium of claim 13, wherein measuring the environmental temperature is performed using a thermometer located at the testing time and at the testing location.

17. The non-transitory machine-readable medium of claim 16, wherein the thermometer is integral with a lateral flow reader.

18. The non-transitory machine-readable medium of claim 16, wherein the thermometer is a wireless thermometer in communication with a lateral flow reader.
